(19) United States
(12) Patent Application Publication
(10) Pub. No.: US 2018/0105555 A1
(43) Pub. Date: Apr. 19, 2018

(54) USE OF DEXTRAN FOR PROTEIN PURIFICATION

(71) Applicant: BRISTOL-MYERS SQUIBB COMPANY, Princeton, NJ (US)

(72) Inventors: Zhijun Tan, Acton, MA (US); Nripen Singh, Acton, MA (US); Mi Jin, West Chester, PA (US); Chao Huang, Shrewsburg, MA (US); Richard P. Martel, JR., Holden, MA (US); Angela T. Lewandowski, Northborough, MA (US); Zhengjian Li, Sudbury, MA (US)

(21) Appl. No.: 15/559,475

(22) PCT Filed: Mar. 18, 2016
(86) PCT No.: PCT/US2016/023092

§ 371 Date: Sep. 19, 2017

Related U.S. Application Data
(60) Provisional application No. 62/136,367, filed on Mar. 20, 2015.

(51) Int. Cl. C07K 1/32; C07K 1/34; C07K 1/36

(52) U.S. Cl. CPC C07K 1/32; C07K 1/36; C07K 1/34

(57) ABSTRACT

In certain embodiments, the invention provides a method of purifying a protein of interest from a mixture which comprises the protein of interest and one or more contaminants, comprising: (a) contacting the mixture with a dextran polymer under conditions suitable for the dextran polymer to bind to one or more contaminants, thereby to form a contaminant precipitate; (b) separating the contaminant precipitate from the mixture to form a solution, thereby purifying the protein of interest.

### CROSS REFERENCE TO RELATED INVENTION

This application claims the benefit of U.S. provisional application Ser. No. 62/136,371 filed Mar. 20, 2015, hereby incorporated by reference in its entirety.

### BACKGROUND OF THE INVENTION

The large-scale, economic purification of proteins is an increasingly important problem for the biopharmaceutical industry. Therapeutic proteins are typically produced using prokaryotic or eukaryotic cell lines that are engineered to express the protein of interest from a recombinant plasmid containing the gene encoding the protein. Separation of the desired protein from the mixture of components fed to the cells and cellular by-products to an adequate purity, e.g., sufficient for use as a human therapeutic, poses a formidable challenge to biologics manufacturers.

Accordingly, there is a need in the art for alternative protein purification methods that can be used to expedite the large-scale processing of protein-based therapeutics, such as antibodies especially due to escalating high titers from cell culture.

### SUMMARY OF THE INVENTION

In certain embodiments, the present invention provides a method of purifying a protein of interest from a mixture which comprises the protein of interest and one or more contaminants, comprising: (a) lowering the pH of the mixture; (b) adding a dextran polymer to the mixture to form a contaminant precipitate; and (c) separating the contaminant precipitate from the mixture to form a solution, thereby purifying the protein of interest. To illustrate, the contaminants are selected from host cell proteins, host cell metabolites, host cell constitutive proteins, nucleic acids, enzymes, endotoxins, viruses, product related contaminants, lipids, media additives and media derivatives, protein aggregates, chromatin, cell culture additives.

In certain specific embodiments, the dextran polymer is selected from dextran, dextran sulfate, dextran sulfate sodium salt, DEAE-dextran hydrochloride. For example, the molecular weight of dextran polymer ranges from 8 kDa to 500 kDa. Optionally, the amount of the dextran polymer is between 0.01 to 0.5% by the volume of the harvest.

In certain specific embodiments, the mixture is selected from a cell culture, a harvested cell culture fluid, a cell culture supernatant, a conditioned cell culture supernatant, a cell lysate, and a clarified bulk. For example, the cell culture is a mammalian cell culture (e.g., a Chinese Hamster Ovary (CHO) cell culture) or a microbial cell culture. Optionally, the cell culture is in a bioreactor.

In certain specific embodiments, the pH of the mixture, before the pH adjustment, is between about 3.0 and about 8.0. Optionally, the pH is lowered before, during or after the addition of the dextran polymer. For example, the pH is lowered by an acid selected from citric acid, acetic acid, and hydrochloric acid. For example, the pH is lowered by at least 1 pH unit. To illustrate, the pH is lowered to a pH ranging from about 3.0 to about 6.5 (e.g., from about 3.0 to about 5.0, or from about 4.0 to about 4.8).

In certain specific embodiments, the contaminant precipitate is separated from the mixture by centrifugation, depth filtration or tangential flow filtration. Optionally, the method further comprises subjecting the post-precipitated solution to a first chromatography (e.g., an ion exchange, hydrophobic interaction, affinity, mimetic, or mixed mode).

In certain specific embodiments, the protein of interest is an antibody or an Fc fusion protein. For example, the protein of interest is a monoclonal antibody (e.g., a human, humanized or chimeric antibody). For example, the protein of interest is substantially in the cell culture supernatant.

In other embodiments, the present invention provides a method of purifying a protein of interest from a mixture which comprises the antibody and one or more contaminants, comprising: (a) contacting the mixture with a dextran polymer under conditions suitable for the dextran polymer to bind to one or more contaminants, thereby to form a contaminant precipitate; (b) separating the contaminant precipitate from the mixture to form a solution, thereby purifying the protein of interest. Optionally, the conditions comprise lowering the pH of the mixture before, during or after the addition of the dextran polymer. In certain specific embodiments, the mixture comprises a feedstock. Optionally, the mixture comprises cell culture media into which the protein of interest is secreted.

In certain specific embodiments, the methods of the present invention can be utilized to reduce the level of one or more contaminants selected from nucleic acids, host cell proteins, and protein aggregates.

### BRIEF DESCRIPTION OF THE DRAWING

FIG. 1 shows pH and dextran sulfate concentration optimization.

FIG. 2 shows impurity levels of clarified bulk and protein A elution pools for antibody A CCF with and without harvest treatment (pH only and pH with dextran sulfate).

FIG. 3 shows fine tuning of pH value for the better recovery and higher impurity removal.

FIG. 4 shows neutralization placement prior to 0.2 um sterile filtration.

FIG. 5 shows product recovery, post treatment turbidity, and impurity levels for neutralization position 1, in which the treated solution was neutralized after mixing and holding at low pH for 60 minutes (*Turbidity was over-range).

FIG. 6 shows product recovery and impurity levels for neutralization scenarios 2 and 3.

FIG. 7 shows recovery, HCP and Monomer in clarified bulk, protein A elution after harvest treatment using dextran sulfate with three different molecular weights.

FIG. 8 shows impurity levels of clarified bulk and protein A elution pools for antibody B CCF with and without harvest treatment (pH only and pH with dextran sulfate).

FIG. 9 shows SEC profiles of protein A elution with (top) and without (bottom) harvest treatment.

FIG. 10 shows fractionation of aggregates from untreated protein A eluate.

FIG. 11 shows that characterization of aggregates from untreated protein A eluate revealed high level of HCP and DNA in the HMW1 species.

FIG. 12 shows chip-based CE-SDS for HMW1 aggregate species.

FIG. 13 shows Trx/TrxR and —SH levels before and after harvest treatment.

### DETAILED DESCRIPTION OF THE INVENTION

In certain aspects, the present invention provides a protein purification method which utilizes acid precipitation/flocculation with dextran sulfate during harvest treatment. Such method can be used as a robust downstream process for purifying proteins, such as monoclonal antibodies.

In certain specific embodiments, the method of the present invention can be utilized for recovering a monoclonal antibody from CHO cell culture, allowing for a robust two-column process. The results demonstrate that the method is not only very effective in reducing impurities (e.g., host cell protein, DNA, and product aggregates), but also significantly improves the depth filter throughput. Additionally, the recovery step has a profound impact on the subsequent downstream process. The process is consistent and robust across a range of harvest treatment and depth filtration operating conditions. An additional study with different harvest feed streams further demonstrates process robustness, while successful pilot-scale runs demonstrate the scalability of the process.

In certain embodiments, the present invention provides a method of purifying a protein of interest from a mixture which comprises the protein of interest and one or more contaminants, comprising: (a) lowering the pH of the mixture; (b) adding a dextran polymer to the mixture to form a contaminant precipitate; and (c) separating the contaminant precipitate from the mixture to form a solution, thereby purifying the protein of interest. To illustrate, the contaminants are selected from host cell proteins, host cell metabolites, host cell constitutive proteins, nucleic acids, enzymes, endotoxins, viruses, product related contaminants, lipids, media additives and media derivatives, protein aggregates, chromatin, cell culture additives.

In certain specific embodiments, the dextran polymer is selected from dextran, dextran sulfate, dextran sulfate sodium salt, DEAE-dextran hydrochloride. For example, the molecular weight of dextran polymer ranges from 8 kDa to 500 kDa. Optionally, the amount of the dextran polymer is between 0.01 to 0.5% by the volume of the harvest.

In certain specific embodiments, the mixture is selected from a cell culture, a harvested cell culture fluid, a cell culture supernatant, a conditioned cell culture supernatant, a cell lysate, and a clarified bulk. For example, the cell culture is a mammalian cell culture (e.g., a Chinese Hamster Ovary (CHO) cell culture) or a microbial cell culture. Optionally, the cell culture is in a bioreactor.

In certain specific embodiments, the pH of the mixture, before the pH adjustment, is between about 3.0 and about 8.0. Optionally, the pH is lowered before, during or after the addition of the dextran polymer. For example, the pH is lowered by an acid selected from citric acid, acetic acid, and hydrochloric acid. For example, the pH is lowered by at least 1 pH unit. To illustrate, the pH is lowered to a pH ranging from about 3.0 to about 6.5 (e.g., from about 3.0 to about 5.0, or from about 4.0 to about 4.8).

In certain specific embodiments, the contaminant precipitate is separated from the mixture by centrifugation, depth filtration or tangential flow filtration. Optionally, the method further comprises subjecting the post-precipitated solution to a first chromatography (e.g., an ion exchange, hydrophobic interaction, affinity, mimetic, or mixed mode).

In certain specific embodiments, the protein of interest is an antibody or an Fc fusion protein. For example, the protein of interest is a monoclonal antibody (e.g., a human, humanized or chimeric antibody). For example, the protein of interest is substantially in the cell culture supernatant.

In other embodiments, the present invention provides a method of purifying a protein of interest from a mixture which comprises the antibody and one or more contaminants, comprising: (a) contacting the mixture with a dextran polymer under conditions suitable for the dextran polymer to bind to one or more contaminants, thereby to form a contaminant precipitate; (b) separating the contaminant precipitate from the mixture to form a solution, thereby purifying the protein of interest. Optionally, the conditions comprise lowering the pH of the mixture before, during or after the addition of the dextran polymer. In certain specific embodiments, the mixture comprises a feedstock. Optionally, the mixture comprises cell culture media into which the protein of interest is secreted.

In certain specific embodiments, the methods of the present invention can be utilized to reduce the level of one or more contaminants selected from nucleic acids, host cell proteins, and protein aggregates.

### I. Definitions

In order that the present disclosure may be more readily understood, certain terms are first defined. As used in this application, except as otherwise expressly provided herein, each of the following terms shall have the meaning set forth below. Additional definitions are set forth throughout the application.

As used herein the term “dextran polymer” refers to dextran or any derivatives or its salt thereof, including, but not limited to, dextran, dextran sulfate, dextran sulfate sodium salt, and DEAE-dextran hydrochloride. For example, the molecular weight of dextran polymer may range from 8 kDa to 500 kDa.

As used herein, the term “protein of interest” is used in its broadest sense to include any protein (either natural or recombinant), present in a mixture, for which purification is desired. Such proteins of interest include, without limitation, hormones, growth factors, cyotokines, immunoglobulins (e.g., antibodies), and immunoglobulin-like domain-containing molecules (e.g., ankyrin or fibronectin domain-containing molecules).

As used herein, a “cell culture” refers to cells in a liquid medium. Optionally, the cell culture is contained in a bioreactor. The cells in a cell culture can be from any organism including, for example, bacteria, fungus, insects, mammals or plants. In a particular embodiment, the cells in a cell culture include cells transfected with an expression construct containing a nucleic acid that encodes a protein of interest (e.g., an antibody). Suitable liquid media include, for example, nutrient media and non-nutrient media. In a particular embodiment, the cell culture comprises a Chinese Hamster Ovary (CHO) cell line in nutrient media, not subject to purification by, for example, filtration or centrifugation.

As used herein, the term “clarified bulk” refers to a mixture from which particulate matter has been substantially removed. Clarified bulk includes cell culture, or cell lysate from which cells or cell debris has been substantially removed by, for example, filtration or centrifugation.

As used herein “bioreactor” takes its art recognized meaning and refers to a chamber designed for the controlled growth of a cell culture. The bioreactor can be of any size as long as it is useful for the culturing of cells, e.g., mammalian cells. Typically, the bioreactor will be at least 30 ml and may be at least 1, 10, 100, 250, 500, 1000, 2500, 5000, 8000, 10,000, 12,0000 liters or more, or any intermediate volume. The internal conditions of the bioreactor, including but not limited to pH and temperature, are typically controlled during the culturing period. A suitable bioreactor may be composed of (i.e., constructed of) any material that is suitable for holding cell cultures suspended in media under the culture conditions and is conductive to cell growth and viability, including glass, plastic or metal; the material(s) should not interfere with expression or stability of a protein of interest. One of ordinary skill in the art will be aware of, and will be able to choose, suitable bioreactors for use in practicing the present invention.

As used herein, a “mixture” comprises a protein of interest (for which purification is desired) and one or more contaminant, i.e., impurities. In one embodiment, the mixture is produced from a host cell or organism that expresses the protein of interest (either naturally or recombinantly). Such mixtures include, for example, cell cultures, cell lysates, and clarified bulk (e.g., clarified cell culture supernatant).

As used herein, the terms “separating” and “purifying” are used interchangeably, and refer to the selective removal of contaminants from a mixture containing a protein of interest (e.g., an antibody). For example, the invention achieves this by precipitation of the contaminants using a dextran polymer in the presence of low pH. Following precipitation, the contaminant precipitate can be removed from the mixture using any means compatible with the present invention, including common industrial methods such as centrifugation or filtration. This separation results in the recovery of a mixture with a substantially reduced level of contaminants, and thereby serves to increase the purity of the protein of interest (e.g., an antibody) in the mixture.

As used herein, the term “contaminant precipitate” refers to an insoluble substance comprising one or more contaminants formed in a solution due to the addition of a compound (e.g., a dextran polymer in the presence of low pH) to the solution.

As used herein the term “contaminant” is used in its broadest sense to cover any undesired component or compound within a mixture. In cell cultures, cell lysates, or clarified bulk (e.g., clarified cell culture supernatant), contaminants include, for example, host cell nucleic acids (e.g., DNA), host cell proteins, host cell metabolites, enzymes, endotoxins, viruses, product related contaminants, lipids, media additives and media derivatives, protein aggregates, chromatin, or cell culture additives. Host cell contaminant proteins include, without limitation, those naturally or recombinantly produced by the host cell, as well as proteins related to or derived from the protein of interest (e.g., proteolytic fragments) and other process related contaminants. In certain embodiments, the contaminant precipitate is separated from the cell culture using an art-recognized means, such as centrifugation, sterile filtration, depth filtration and tangential flow filtration.

As used herein “centrifugation” is a process that involves the use of the centrifugal force for the sedimentation of heterogeneous mixtures with a centrifuge, used in industry and in laboratory settings. This process is used to separate two immiscible liquids. For example, in a method of the present invention, centrifugation can be used to remove a contaminant precipitation from a mixture, including without limitation, a cell culture or clarified cell culture supernatant or capture-column captured elution pool.

As used herein “sterile filtration” is a filtration method that use membrane filters, which are typically a filter with pore size 0.2 μm to effectively remove microorganisms or small particles. For example, in a method of the present invention, sterile filtration can be used to remove a contaminant precipitate from a mixture, including without limitation, a cell culture or clarified cell culture supernatant or capture-column captured elution pool.

As used herein “depth filtration” is a filtration method that uses depth filters, which are typically characterized by their design to retain particles due to a range of pore sizes within a filter matrix. The depth filter's capacity is typically defined by the depth, e.g., 10 inch or 20 inch of the matrix and thus the holding capacity for solids. For example, in a method of the present invention, depth filtration can be used to remove a contaminant precipitate from a mixture, including without limitation, a cell culture or clarified cell culture supernatant or capture-column captured elution pool.

As used herein, the term “tangential flow filtration” refers to a filtration process in which the sample mixture circulates across the top of a membrane, while applied pressure causes certain solutes and small molecules to pass through the membrane. For example, in a method of the present invention, tangential flow filtration can be used to remove a contaminant precipitate from a mixture, including without limitation, a cell culture or clarified cell culture supernatant or capture-column captured elution pool.

As used herein the term “chromatography” refers to the process by which a solute of interest, e.g., a protein of interest, in a mixture is separated from other solutes in the mixture by percolation of the mixture through an adsorbent, which adsorbs or retains a solute more or less strongly due to properties of the solute, such as pI, hydrophobicity, size and structure, under particular buffering conditions of the process. In a method of the present invention, chromatography can be used to remove contaminants after the precipitate is removed from a mixture, including without limitation, a cell culture or clarified cell culture supernatant or capture-column captured elution pool.

The term “affinity chromatography” refers to a chromatographic method in which a biomolecule such as a polypeptide is separated based on a specific reversible interaction between the polypeptide and a binding partner covalently coupled to the solid phase. Examples of affinity interactions include, but are not limited to, the reversible interaction between an antigen and antibody, enzyme and substrate, or receptor and ligand. In certain specific embodiments, affinity chromatography involves the use of microbial proteins, such as Protein A or Protein G. Protein A is a bacterial cell wall protein that binds to mammalian IgGs primarily through their Fc regions. Protein A resin is useful for affinity purification and isolation of a variety antibody isotypes, particularly IgG1, IgG2, and IgG4. There are many Protein A resins available that are suitable for use in the purification process described herein. The resins are generally classified based on their backbone composition and include, for example, glass or silica-based resins; agarose-based resins; and organic polymer based resins.

The terms “ion-exchange” and “ion-exchange chromatography” refer to a chromatographic process in which an ionizable solute of interest (e.g., a protein of interest in a mixture) interacts with an oppositely charged ligand linked (e.g., by covalent attachment) to a solid phase ion exchange material under appropriate conditions of pH and conductivity, such that the solute of interest interacts non-specifically with the charged compound more or less than the solute impurities or contaminants in the mixture. The contaminating solutes in the mixture can be washed from a column of the ion exchange material or are bound to or excluded from the resin, faster or slower than the solute of interest. “Ion-exchange chromatography” specifically includes cation exchange, anion exchange, and mixed mode chromatographies.

The phrase “ion exchange material” refers to a solid phase that is negatively charged (i.e., a cation exchange resin or membrane) or positively charged (i.e., an anion exchange resin or membrane). In one embodiment, the charge can be provided by attaching one or more charged ligands (or adsorbents) to the solid phase, e.g., by covalent linking. Alternatively, or in addition, the charge can be an inherent property of the solid phase (e.g., as is the case for silica, which has an overall negative charge).

A “cation exchange resin” refers to a solid phase which is negatively charged, and which has free cations for exchange with cations in an aqueous solution passed over or through the solid phase. Any negatively charged ligand attached to the solid phase suitable to form the cation exchange resin can be used, e.g., a carboxylate, sulfonate and others as described below. Commercially available cation exchange resins include, but are not limited to, for example, those having a sulfonate based group (e.g., MonoS, MiniS, Source 15S and 30S, SP Sepharose Fast Flow′, SP Sepharose High Performance from GE Healthcare, Toyopearl SP-650S and SP-650M from Tosoh, Macro-Prep High S from BioRad, Ceramic HyperD S, Trisacryl M and LS SP and Spherodex LS SP from Pall Technologies); a sulfoethyl based group (e.g., Fractogel SE, from EMD, Poros S-10 and S-20 from Applied Biosystems); a sulphopropyl based group (e.g., TSK Gel SP 5PW and SP-5PW-HR from Tosoh, Poros HS-20 and HS 50 from Applied Biosystems); a sulfoisobutyl based group (e.g., Fractogel EMD SO₃⁻ from EMD); a sulfoxyethyl based group (e.g., SE52, SE53 and Express-Ion S from Whatman), a carboxymethyl based group (e.g., CM Sepharose Fast Flow from GE Healthcare, Hydrocell CM from Biochrom Labs Inc., Macro-Prep CM from BioRad, Ceramic HyperD CM, Trisacryl M CM, Trisacryl LS CM, from Pall Technologies, Matrx Cellufine C500 and C200 from Millipore, CM52, CM32, CM23 and Express-Ion C from Whatman, Toyopearl CM-650S, CM-650M and CM-650C from Tosoh); sulfonic and carboxylic acid based groups (e.g., BAKERBOND Carboxy-Sulfon from J.T. Baker); a carboxylic acid based group (e.g., WP CBX from J.T Baker, DOWEX MAC-3 from Dow Liquid Separations, Amberlite Weak Cation Exchangers, DOWEX Weak Cation Exchanger, and Diaion Weak Cation Exchangers from Sigma-Aldrich and Fractogel EMD COO— from EMD); a sulfonic acid based group (e.g., Hydrocell SP from Biochrom Labs Inc., DOWEX Fine Mesh Strong Acid Cation Resin from Dow Liquid Separations, UNOsphere S, WP Sulfonic from J. T. Baker, Sartobind S membrane from Sartorius, Amberlite Strong Cation Exchangers, DOWEX Strong Cation and Diaion Strong Cation Exchanger from Sigma-Aldrich); and a orthophosphate based group (e.g., P11 from Whatman).

An “anion exchange resin” refers to a solid phase which is positively charged, thus having one or more positively charged ligands attached thereto. Any positively charged ligand attached to the solid phase suitable to form the anionic exchange resin can be used, such as quaternary amino groups Commercially available anion exchange resins include DEAE cellulose, Poros PI 20, PI 50, HQ 10, HQ 20, HQ 50, D 50 from Applied Biosystems, Sartobind Q from Sartorius, MonoQ, MiniQ, Source 15Q and 30Q, Q, DEAE and ANX Sepharose Fast Flow, Q Sepharose high Performance, QAE SEPHADEX™ and FAST Q SEPHAROSE™ (GE Healthcare), WP PEI, WP DEAM, WP QUAT from J.T. Baker, Hydrocell DEAE and Hydrocell QA from Biochrom Labs Inc., UNOsphere Q, Macro-Prep DEAE and Macro-Prep High Q from Biorad, Ceramic HyperD Q, ceramic HyperD DEAE, Trisacryl M and LS DEAE, Spherodex LS DEAE, QMA Spherosil LS, QMA Spherosil M and Mustang Q from Pall Technologies, DOWEX Fine Mesh Strong Base Type I and Type II Anion Resins and DOWEX MONOSPHER E 77, weak base anion from Dow Liquid Separations, Intercept Q membrane, Matrex Cellufine A200, A500, Q500, and Q800, from Millipore, Fractogel EMD TMAE, Fractogel EMD DEAE and Fractogel EMD DMAE from EMD, Amberlite weak strong anion exchangers type I and II, DOWEX weak and strong anion exchangers type I and II, Diaion weak and strong anion exchangers type I and II, Duolite from Sigma-Aldrich, TSK gel Q and DEAE 5PW and 5PW-HR, Toyopearl SuperQ-650S, 650M and 650C, QAE-550C and 650S, DEAE-650M and 650C from Tosoh, QA52, DE23, DE32, DE51, DE52, DE53, Express-Ion D and Express-Ion Q from Whatman, and Sartobind Q (Sartorius corporation, New York, USA).

A “mixed mode ion exchange resin” or “mixed mode” refers to a solid phase which is covalently modified with cationic, anionic, and/or hydrophobic moieties. Examples of mixed mode ion exchange resins include BAKERBOND ABX™ (J. T. Baker; Phillipsburg, N.J.), ceramic hydroxyapatite type I and II and fluoride hydroxyapatite (BioRad; Hercules, Calif.) and MEP and MBI HyperCel (Pall Corporation; East Hills, N.Y.).

A “hydrophobic interaction chromatography resin” refers to a solid phase which is covalently modified with phenyl, octyl, or butyl chemicals. Hydrophobic interaction chromatography is a separation technique that uses the properties of hydrophobicity to separate proteins from one another. In this type of chromatography, hydrophobic groups such as, phenyl, octyl, or butyl are attached to the stationary column. Proteins that pass through the column that have hydrophobic amino acid side chains on their surfaces are able to interact with and bind to the hydrophobic groups on the column. Examples of hydrophobic interaction chromatography resins include Phenyl sepharose FF, Capto Phenyl (GE Healthcare, Uppsala, Sweden), Phenyl 650-M (Tosoh Bioscience, Tokyo, Japan) and Sartobind Phenyl (Sartorius corporation, New York, USA).

### II. Proteins of Interest

In certain aspects, methods of the present invention may be used to purify any protein of interest including, but not limited to, proteins having pharmaceutical, diagnostic, agricultural, and/or any of a variety of other properties that are useful in commercial, experimental or other applications. In addition, a protein of interest can be a protein therapeutic. In certain embodiments, proteins purified using methods of the present invention may be processed or modified. For example, a protein of interest in accordance with the present invention may be glycosylated.

Thus, the present invention may be used to culture cells for production of any therapeutic protein, such as pharmaceutically or commercially relevant enzymes, receptors, receptor fusion proteins, antibodies (e.g., monoclonal or polyclonal antibodies), antigen-binding fragments of an antibody, Fc fusion proteins, cytokines, hormones, regulatory factors, growth factors, coagulation/clotting factors, or antigen-binding agents. The above list of proteins is merely exemplary in nature, and is not intended to be a limiting recitation. One of ordinary skill in the art will know that other proteins can be produced in accordance with the present invention, and will be able to use methods disclosed herein to produce such proteins.

In one particular embodiment of the invention, the protein purified using the method of the invention is an antibody. The term “antibody” is used in the broadest sense to cover monoclonal antibodies (including full length monoclonal antibodies), polyclonal antibodies, multispecific antibodies (e.g., bispecific antibodies), antibody fragments, immunoadhesins and antibody-immunoadhesin chimerias.

An “antibody fragment” includes at least a portion of a full length antibody and typically an antigen binding or variable region thereof. Examples of antibody fragments include Fab, Fab′, F(ab′)₂, and Fv fragments; single-chain antibody molecules; diabodies; linear antibodies; and multispecific antibodies formed from engineered antibody fragments.

The term “monoclonal antibody” is used in the conventional sense to refer to an antibody obtained from a population of substantially homogeneous antibodies such that the individual antibodies comprising the population are identical except for possible naturally occurring mutations that may be present in minor amounts. Monoclonal antibodies are highly specific, being directed against a single antigenic site. This is in contrast with polyclonal antibody preparations which typically include varied antibodies directed against different determinants (epitopes) of an antigen, whereas monoclonal antibodies are directed against a single determinant on the antigen. The term “monoclonal”, in describing antibodies, indicates the character of the antibody as being obtained from a substantially homogeneous population of antibodies, and is not to be construed as requiring production of the antibody by any particular method. For example, monoclonal antibodies used in the present invention can be produced using conventional hybridoma technology first described by Kohler et al., Nature 256:495 (1975), or they can be made using recombinant DNA methods (see, e.g., U.S. Pat. No. 4,816,567). Monoclonal antibodies can also be isolated from phage antibody libraries, e.g., using the techniques described in Clackson et al., Nature 352:624-628 (1991); Marks et al., J. Mol. Biol. 222:581-597 (1991); and U.S. Pat. Nos. 5,223,409; 5,403,484; 5,571,698; 5,427,908 5,580,717; 5,969,108; 6,172,197; 5,885,793; 6,521,404; 6,544,731; 6,555,313; 6,582,915; and 6,593,081).

The monoclonal antibodies described herein include “chimeric” and “humanized” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA 81:6851-6855 (1984)). “Humanized” forms of non-human (e.g., murine) antibodies are chimeric antibodies which contain minimal sequence derived from non-human immunoglobulin. For the most part, humanized antibodies are human immunoglobulins (recipient antibody) in which the hypervariable region residues of the recipient are replaced by hypervariable region residues from a non-human species (donor antibody) such as mouse, rat, rabbit or nonhuman primate having the desired specificity, affinity, and capacity. In some instances, Fv framework region (FR) residues of the human immunoglobulin are replaced by corresponding non-human residues. Furthermore, humanized antibodies may comprise residues which are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

Chimeric or humanized antibodies can be prepared based on the sequence of a murine monoclonal antibody prepared as described above. DNA encoding the heavy and light chain immunoglobulins can be obtained from the murine hybridoma of interest and engineered to contain non-murine (e.g., human) immunoglobulin sequences using standard molecular biology techniques. For example, to create a chimeric antibody, the murine variable regions can be linked to human constant regions using methods known in the art (see e.g., U.S. Pat. No. 4,816,567 to Cabilly et al.). To create a humanized antibody, the murine CDR regions can be inserted into a human framework using methods known in the art (see e.g., U.S. Pat. No. 5,225,539 to Winter, and U.S. Pat. Nos. 5,530,101; 5,585,089; 5,693,762 and 6,180,370 to Queen et al.).

The monoclonal antibodies described herein also include “human” antibodies, which can be isolated from various sources, including, e.g., from the blood of a human patient or recombinantly prepared using transgenic animals. Examples of such transgenic animals include KM-Mouse® (Medarex, Inc., Princeton, N.J.) which has a human heavy chain transgene and a human light chain transchromosome (see WO 02/43478), Xenomouse® (Abgenix, Inc., Fremont Calif.; described in, e.g., U.S. Pat. Nos. 5,939,598; 6,075,181; 6,114,598; 6, 150,584 and 6,162,963 to Kucherlapati et al.), and HuMAb-Mouse® (Medarex, Inc.; described in, e.g., Taylor, L. et al. (1992) Nucleic Acids Research 20:6287-6295; Chen, J. et al. (1993) International Immunology 5: 647-656; Tuaillon et al. (1993) Proc. Natl. Acad. Sci. USA 90:3720-3724; Choi et al. (1993) Nature Genetics 4:117-123; Chen, J. et al. (1993) EMBO J. 12: 821-830; Tuaillon et al. (1994) J. Immunol. 152:2912-2920; Taylor, L. et al. (1994) International Immunology 6: 579-591; and Fishwild, D. et al. (1996) Nature Biotechnology 14: 845-851, U.S. Pat. Nos. 5,545,806; 5,569,825; 5,625,126; 5,633,425; 5,789,650; 5,877,397; 5,661,016; 5,814,318; 5,874,299; and U.S. Pat. Nos. 5,770,429; 5,545,807; and PCT Publication Nos. WO 92/03918, WO 93/12227, WO 94/25585, WO 97/13852, WO 98/24884 and WO 99/45962, WO 01/14424 to Korman et al.). Human monoclonal antibodies of the invention can also be prepared using SCID mice into which human immune cells have been reconstituted such that a human antibody response can be generated upon immunization. Such mice are described in, for example, U.S. Pat. Nos. 5,476,996 and 5,698,767 to Wilson et al.

### III. Mixtures Containing a Protein of Interest

The methods of the invention can be applied to any mixture containing a protein of interest. In one embodiment, the mixture is obtained from or produced by living cells that express the protein to be purified (e.g., naturally or by genetic engineering). Optionally, the cells in a cell culture include cells transfected with an expression construct containing a nucleic acid that encodes a protein of interest. Methods of genetically engineering cells to produce proteins are well known in the art. See e.g., Ausabel et al., eds. (1990), Current Protocols in Molecular Biology (Wiley, New York) and U.S. Pat. Nos. 5,534,615 and 4,816,567, each of which are specifically incorporated herein by reference. Such methods include introducing nucleic acids that encode and allow expression of the protein into living host cells. These host cells can be bacterial cells, fungal cells, insect cells or, preferably, animal cells grown in culture. Bacterial host cells include, but are not limited to E. coli cells. Examples of suitable E. coli strains include: HB101, DH5α, GM2929, JM109, KW251, NM538, NM539, and any E. coli strain that fails to cleave foreign DNA. Fungal host cells that can be used include, but are not limited to, Saccharomyces cerevisiae, Pichia pastoris and Aspergillus cells. Insect cells that can be used include, but are not limited to, Bombyx mori, Mamestra drassicae, Spodoptera frugiperda, Trichoplusia ni, Drosophilia melanogaster.

A number of mammalian cell lines are suitable host cells for expression of proteins of interest. Mammalian host cell lines include, for example, COS, PER.C6, TM4, VERO076, DXB11, MDCK, BRL-3A, W138, Hep G2, MMT, MRC 5, FS4, CHO, 293T, A431, 3T3, CV-1, C3H10T1/2, Colo205, 293, HeLa, L cells, BHK, HL-60, FRhL-2, U937, HaK, Jurkat cells, Rat2, BaF3, 32D, FDCP-1, PC12, M1x, murine myelomas (e.g., SP2/0 and NS0) and C2C12 cells, as well as transformed primate cell lines, hybridomas, normal diploid cells, and cell strains derived from in vitro culture of primary tissue and primary explants. New animal cell lines can be established using methods well known by those skilled in the art (e.g., by transformation, viral infection, and/or selection). Any eukaryotic cell that is capable of expressing the protein of interest may be used in the disclosed cell culture methods. Numerous cell lines are available from commercial sources such as the American Type Culture Collection (ATCC). In one embodiment of the invention, the cell culture, e.g., the large-scale cell culture, employs hybridoma cells. The construction of antibody-producing hybridoma cells is well known in the art. In one embodiment of the invention, the cell culture, e.g., the large-scale cell culture, employs CHO cells to produce the protein of interest such as an antibody (see, e.g., WO 94/11026). Various types of CHO cells are known in the art, e.g., CHO-K1, CHO-DG44, CHO-DXB11, CHO/dhfr⁻ and CHO—S.

In a specific embodiment, methods of the present invention comprise effectively removing contaminants from a mixture (e.g., a cell culture, cell lysate or clarified bulk) which contains a high concentration of a protein of interest (e.g., an antibody). For example, the concentration of a protein of interest may range from about 0.5 to about 50 mg/ml (e.g., 0.5, 1, 5, 10, 15, 20, 25, 30, 35, 40, 45 or 50 mg/ml).

Preparation of mixtures initially depends on the manner of expression of the protein. Some cell systems directly secrete the protein (e.g., an antibody) from the cell into the surrounding growth media, while other systems retain the antibody intracellularly. For proteins produced intracellularly, the cell can be disrupted using any of a variety of methods, such as mechanical shear, osmotic shock, and enzymatic treatment. The disruption releases the entire contents of the cell into the homogenate, and in addition produces subcellular fragments which can be removed by centrifugation or by filtration. A similar problem arises, although to a lesser extent, with directly secreted proteins due to the natural death of cells and release of intracellular host cell proteins during the course of the protein production run.

In one embodiment, cells or cellular debris are removed from the mixture, for example, to prepare clarified bulk. The methods of the invention can employ any suitable methodology to remove cells or cellular debris. If the protein is produced intracellularly, as a first step, the particulate debris, either host cells or lysed fragments, can be removed, for example, by a centrifugation or filtration step in order to prepare a mixture which is then subjected to purification according the methods described herein (i.e., from which a protein of interest is purified). If the protein is secreted into the medium, the recombinant host cells may be separated from the cell culture medium by, e.g., centrifugation, tangential flow filtration or depth filtration, in order to prepare a mixture from which a protein of interest is purified.

In another embodiment, cell culture or cell lysate is used directly without first removing the host cells. Indeed, the methods of the invention are particularly well suited to using mixtures comprising a secreted protein and a suspension of host cells.

### IV. Contaminants Precipitation by a Dextran Polymer at Low pH

In certain embodiments, methods of the present invention involve (a) contacting a mixture with a dextran polymer under conditions suitable for the dextran polymer to bind to one or more contaminants, thereby to form a contaminant precipitate; (b) separating the contaminant precipitate from the mixture to form a solution, thereby purifying the protein of interest. For example, the conditions comprise lowering the pH of the mixture before, during or after the addition of the dextran polymer.

To illustrate, the dextran polymer may be selected from dextran, dextran sulfate, dextran sulfate sodium salt, DEAE-dextran hydrochloride. For example, the molecular weight of dextran polymer ranges from 8 kDa to 500 kDa.

Preferably, the pH of the mixture is adjusted to facilitate precipitation. The optimum pH required to facilitate precipitation of a particular contaminant can be determined empirically for each protein mixture using methods described herein. For example, the pH of the mixture, before the pH adjustment, is between about 3.0 and about 8.0. Optionally, the pH of the mixture is lowered before, during or after the addition of the dextran polymer. Preferably, the pH is lowered by at least 1 pH unit. To illustrate, the pH is lowered to a pH ranging from about 3.0 to about 6.5, from about 3.0 to about 5.0, or from about 4.0 to about 4.8 (e.g., 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0). In general, any art recognized acids or buffers can be used to alter the pH of a mixture, including, for example, citric acid, acetic acid, hydrochloric acid, acetate- or citrate-containing buffers. An advantage of using a bioreactor cell culture is that the pH of the cell culture medium can be monitored and adjusted by addition of one or more suitable acids or buffers to the cell culture medium in the bioreactor.

To illustrate, the contaminant precipitate is separated from the mixture by centrifugation, depth filtration or tangential flow filtration. Optionally, the method further comprises subjecting the post-precipitated solution to a first chromatography (e.g., an ion exchange, hydrophobic interaction, affinity, mimetic, or mixed mode).

The concentration of the dextran polymer sufficient to precipitate contaminants from a particular mixture can be determined empirically for each protein mixture using methods described herein. The final concentration of the dextran polymer added to the mixture is at least 0.01% by the volume of the harvest (w/v), for example between about 0.01% and 0.5% (w/v), such as 0.01%, 0.02%, 0.03%, 0.04%, 0.05%, 0.06%, 0.07%, 0.08%, 0.09%, 0.1%, 0.2%, 0.3%, 0.4%, or 0.5 (w/v).

In certain embodiments, the dextran polymer is added to the mixture and mixed for a particular length of time prior to removing the contaminant precipitate. The optimum length of mixing required to facilitate precipitation of a particular contaminant can be determined empirically for each protein mixture using methods described herein. Preferably the mixing time is greater than about 5 minutes (e.g., about 5, 10, 15, 20, 30, 60, 90, 120, 240, or 480 minutes). In a particular embodiment, the mixing time is about 60 minutes.

The present disclosure is further illustrated by the following examples, which should not be construed as further limiting. The contents of all figures and all references, patents and published patent applications cited throughout this application are expressly incorporated herein by reference in their entireties.

### Example 1

### pH Values and Dextran Sulfate Concentrations

This experiment compared different pH values and dextran sulfate concentrations for acid precipitation for CHO cell culture and compared their contaminant removal levels.

CHO cells expressing a recombinant monoclonal antibody were grown in a fed batch culture for 14 days. Cell culture fluid (CCF) was cooled to room temperature prior to experiment. Both citric acid (CAS No. 5949-29) and dextran sulfate (500 kDa, Product No. 31392) were purchased from Sigma (St. Louis, Mo.). 1M citric acid and 10 g/L dextran sulfate working solution used in all experiments were prepared by dissolving into water. The CCF was adjusted to a studied pH with citric acid followed by gradual addition of the 10 g/L dextran sulfate working solution to different target concentrations. The solution was stirred for at 60 minutes at room temperature.

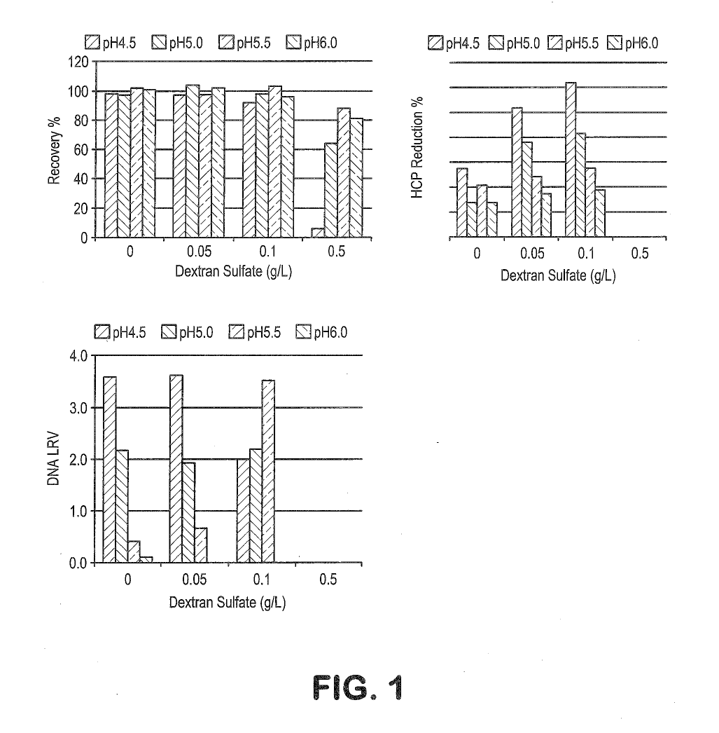

The flocculated solutions were then centrifuged at 500×g for 10 minutes to remove precipitates. The supernatants were passed through 0.2 um filters and were tested for product recovery and impurity levels including host cell protein, DNA. As depicted in FIG. 1, adjusted pH range of 4.5 to 5.5 for CCF and target dextran sulfate concentration of 0.1 g/L appear to be an optimal treatment condition in terms of high product recovery and sound HCP and DNA removal.

### Harvest Treatment Followed by Protein a Chromatography

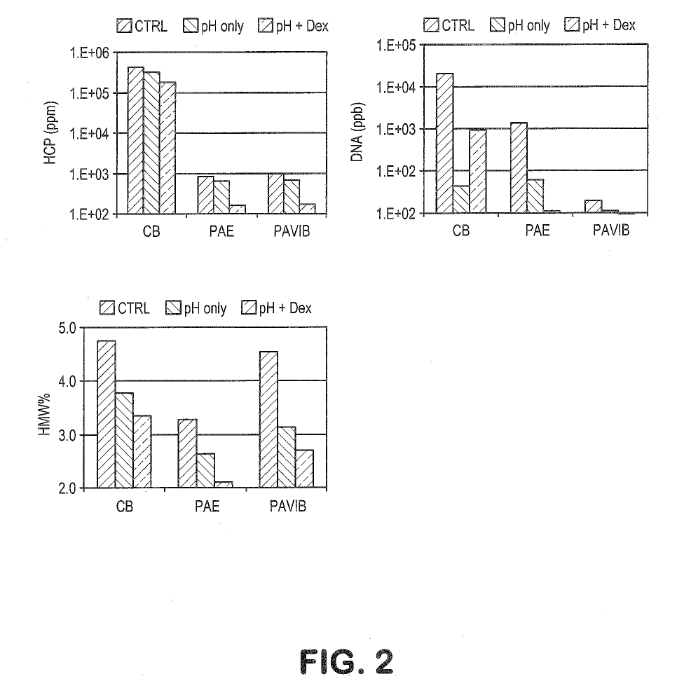

The optimal harvest treatment condition was further evaluated by analyzing impurity levels in protein A samples. Three clarified bulk (CB) samples (untreated CB, low pH treated CB, and low pH with dextran sulfate treated CB) were purified with protein A chromatographic column using MabSelect SuRe resin (GE Healthcare, Uppsala, Sweden). The protein A elution samples as well as the initial CB samples were analyzed for HCP, DNA and HMW, as depicted in FIG. 2. The harvest treatment using acid precipitation coupled with dextran sulfate was proven to be very effective in reducing HCP, DNA and HMW species. Interestingly, the HCP values in CB samples seemed little changed by the treatment, suggesting that low pH with dextran sulfate treatment in particular removed those HCP species, which otherwise would be the most challenging to be removed by protein A column.

### Example 2

### Fine Tune of pH for Harvest Treatment

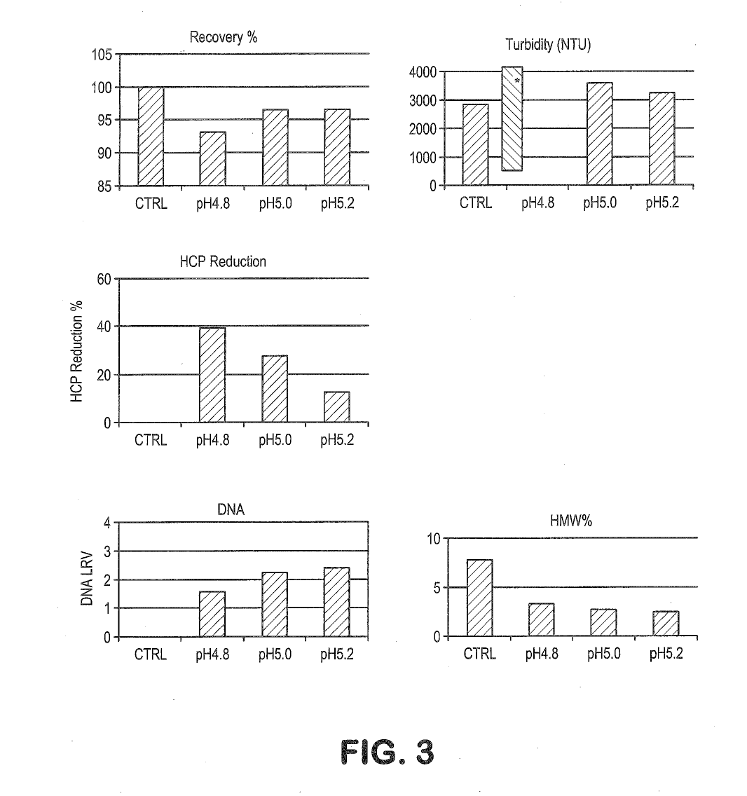

This experiment further optimized the harvest treatment pH. A narrower pH range (center point ±0.1) was defined. Previously the pH range was 4.5 to 5.5, which is still wide from operational perspective. In this experiment, the dextran sulfate concentration in the CCF was constant at 0.1 g/L and the pH was varied from 4.8 to 5.2. The product recovery, CCF turbidity, HCP, DNA and aggregates in the clarified CB were evaluated and shown in FIG. 3. Although the pH from 4.8 to 5.2 seems to be a narrow range, the impact on impurity removal is pronounced. The treatment condition at pH 4.8 clearly shows much better HCP and DNA reduction with acceptable product recovery (>90%). Interestingly, the pH4.8 treated CCF showed highest turbidity, suggesting highest impurity removal by precipitation.

### Example 3

### Placement of Neutralization Prior to 0.2 um Sterile Filtration

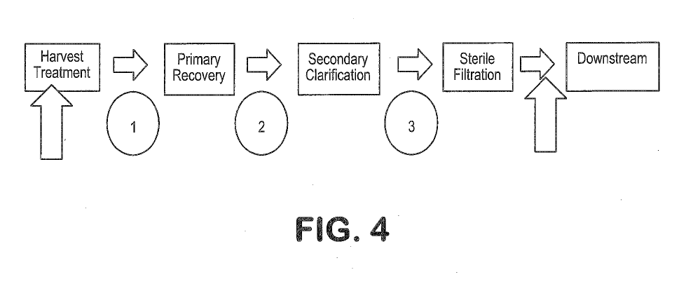

This experiment decides the position of neutralization of the low pH treated solution before becoming final clarified bulk, which is at neutral pH prior to the protein A column step. There are several possible scenarios as shown in FIG. 4. Each scenario has its pros and cons, as listed in Table 1.

TABLE 1
Pros and cons for neutralization placement positions

| Position | Detail | Rationale | Concerns |
|---|---|---|---|
| 1 | Neutralize in | Convenience | Poor impurity |
| | bioreactor | | removal |
| 2 | Neutralize prior | Potential better | Manufacturability, |
| | to secondary | impurity removal | product recovery |
| | clarification | | |
| 3 | Neutralize prior | Continuous filtration | Impurity removal for |
| | to 0.2 μm sterile | | 2^{nd} stage filter may |
| | filtration | | not be ideal |

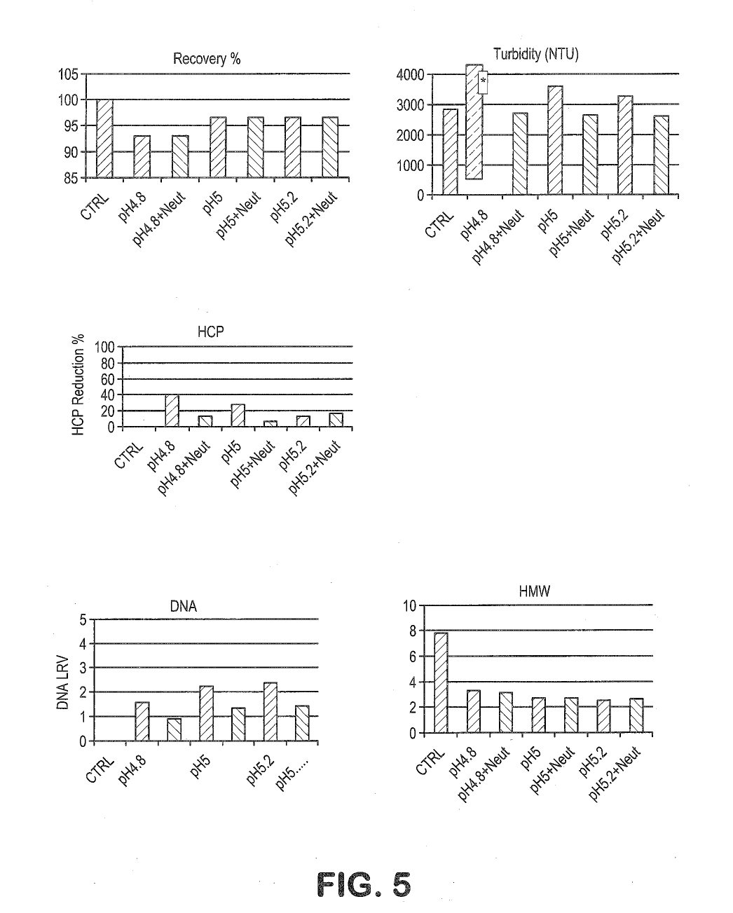

For scenario 1, Applicants first performed harvest treatment at low pH with dextran sulfate. Several pH conditions were chosen for the study. Then Applicants adjusted pH back to neutral condition with 1-2M tris. The product recovery, CCF turbidity, and levels of HCP, DNA and aggregation before and after neutralization were compared. As shown in FIG. 5, after neutralization the turbidity of the treated CCF decreased and HCP and DNA in the supernatant increased, suggesting that some HCP and DNA went back to the solution. Therefore the scenario 1 is not an ideal option.

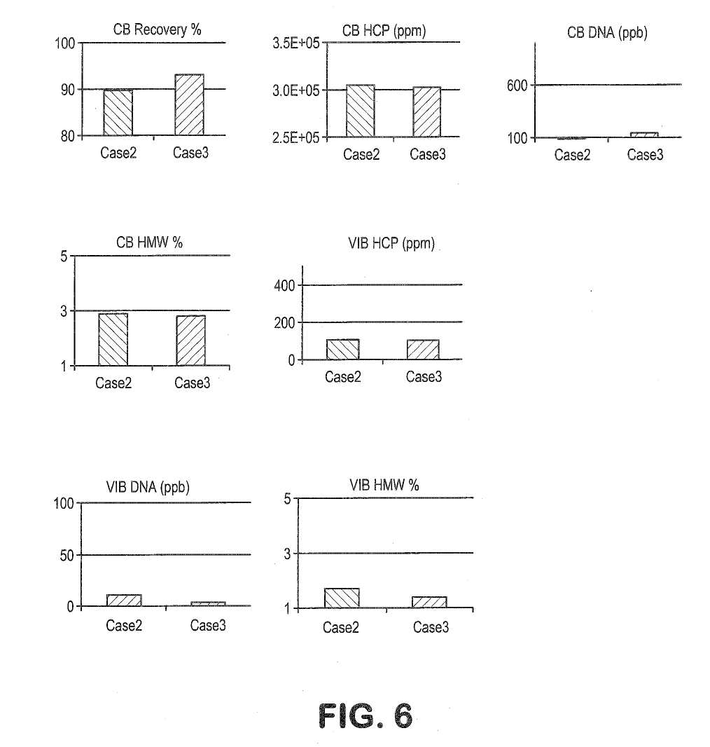

The product recovery and impurity levels of CB samples for neutralization scenarios 2 and 3 were presented in FIG. 6. Both CB samples were purified with protein A column and HCP, DNA and HMW were analyzed. As shown in FIG. 6, it appeared that scenario 2 and 3 had comparable impurity levels in CB solutions and protein A elution samples, but scenario 3 has slight edge in product recovery. By taking the consideration of the better manufacturability of scenario 3, the treated solution was neutralized after the secondary clarification and prior to 0.2 um sterile filtration.

### Example 4

### Impact of Dextran Sulfate with Different MWs on Impurity Removal

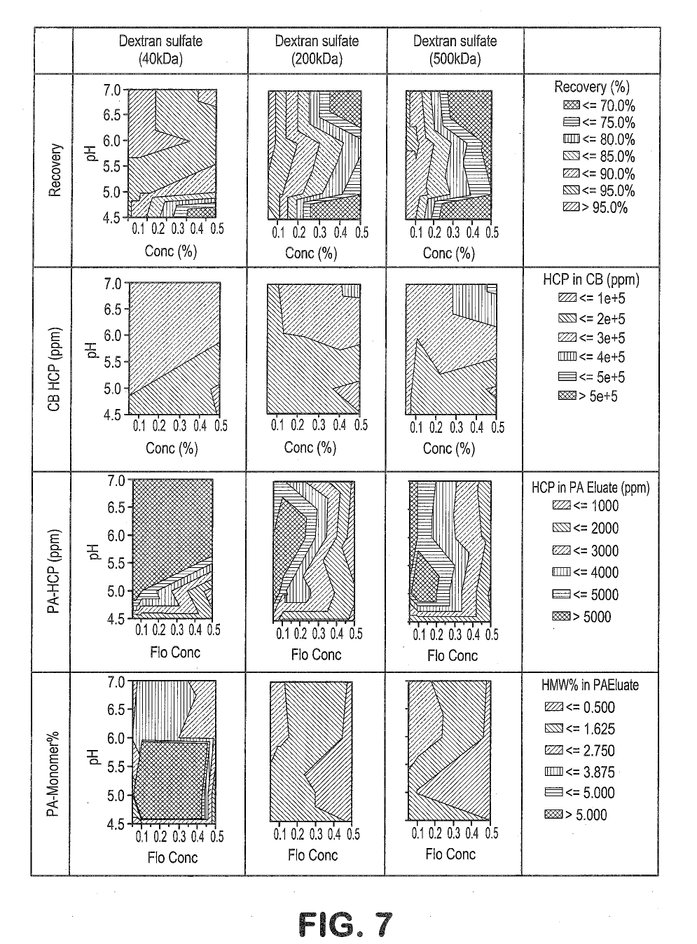

This experiment compared dextran sulfates with different molecular weights (MW) in harvest treatment effectiveness in removing impurities. A high throughput plate format was used for this particular study. The CCF pH, dextran sulfate concentration, and dextran sulfate MWs were evaluated. The 40 kDa dextran sulfate (CAS No. 9011-18) was purchased from Spectrum Chemicals (New Brunswick, N.J.) and the 200 kDa dextran sulfate (CAS No. 67578) was purchased from Sigma. The product recovery, HCP in the clarified bulk, HCP and monomer % in the protein A elution were plotted and shown in FIG. 7. All three dextran sulfate showed similar results in terms of recovery, HCP removal and monomer purity.

### Example 5

### Impact of Extended Cell Culture on Impurity Removal (Day 14 vs. Day 17)

This experiment evaluated the effectiveness of the harvest treatment in impurity removal for the worst-case scenario of cell culture. This CHO cell culture normally is harvested in 14 days. For this particular study, the harvest of two batches of cell culture material was extended to 17 days to generate the worst-case cell culture with 10-20 percentage lower viability than day 14 cell culture. All four CB samples and protein A purified samples were analyzed for impurity levels and results were summarized in Table 2. It appeared that prolonged cell culture did not pose significant challenge in terms of impurity clearance. In other words, the harvest treatment using low pH coupled with dextran sulfate was capable of removing impurities from cell culture with extreme low viability with high impurity levels.

TABLE 2
Results of extended cell culture with harvest treatment

| | | X2 Day14 | X2 Day17 | X3 Day14 | X3 Day17 |
|---|---|---|---|---|---|
| Upstream | Viability % | 50.2 | 31.2 | 51.2 | 42 |
| CB | HCP (ppm) | 1.4E4 | 1.7E5 | 1.3E3 | 1.7E5 |
| | DNA (ppb) | <1000 | <100 | <1000 | 38 |
| | HMW % | 3.4 | 3.0 | 3.4 | 2.9 |
| PAVIB | HCP (ppm) | 350 | 380 | 460 | 230 |
| | HMW % | 4.2 | 4.0 | 4.1 | 4.3 |

### Example 6

### Impact of Different Processes on Impurity Removal (A vs. B)

This experiment evaluated the effectiveness of impurity removal by harvest treatment for the cell culture materials from two different processes, which have different cell densities and possibly different impurity profiles. The two cell cultures underwent low pH and dextran sulfate treatment and the CB solutions were purified with protein A column. The protein A elution samples were analyzed for HCP and DNA and results were summarized in Table 3. The impurity levels in both processes are acceptable, indicating the harvest treatment is capable of removing impurities effectively regardless of potential variability of initial impurity profile or impurity levels as resulted from different processes.

TABLE 3
Results of protein A elutions from two different
processes with harvest treatment

| | | Process A | Process B |
|---|---|---|---|
| | | (Day17) | (Day14) |
| Upstream | Viability % | 52.4 | 69 |
| | Cell Density (10\hat{ }6/mL) | 5 | 14 |
| PAVIB | HCP (ppm) | 70 | 367 |
| | DNA (ppb) | 0.2 | <DT |
| | HMW % | 2.1 | 4.4 |

### Example 7

### Impact of Harvest Treatment on Antibody B

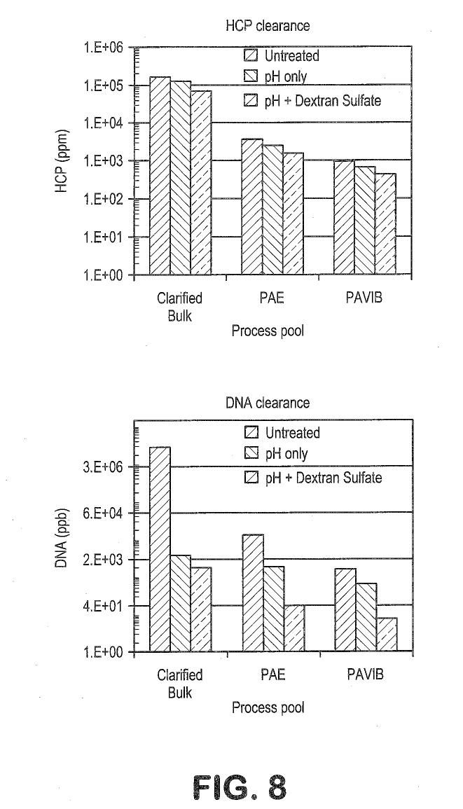

This experiment evaluated the effectiveness of impurity removal for monoclonal antibody B using the same harvest treatment conditions. The cell culture was treated with two conditions: low pH only at 4.8 and low pH with addition of dextran sulfate with the final concentration of 0.1 g/L. The treated CCF was mixed at room temperature for minimum 60 minutes followed by centrifugation and 0.2 μm filter. The clarified bulk and post protein A eluates were tested for HCP and DNA. As shown in FIG. 8, the low treatment coupled with dextran sulfate was effective in removing HCP and DNA from

### Example 8

### Chromatin Removal

This example demonstrated that the harvest treatment was capable of removing chromatin from the antibody cell culture by acid precipitation with dextran sulfate.

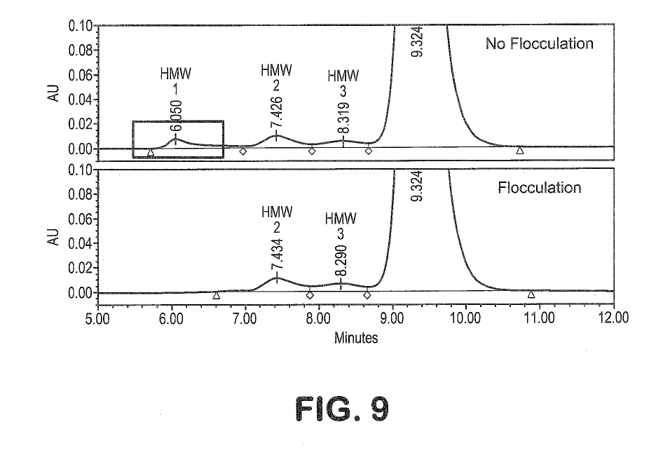

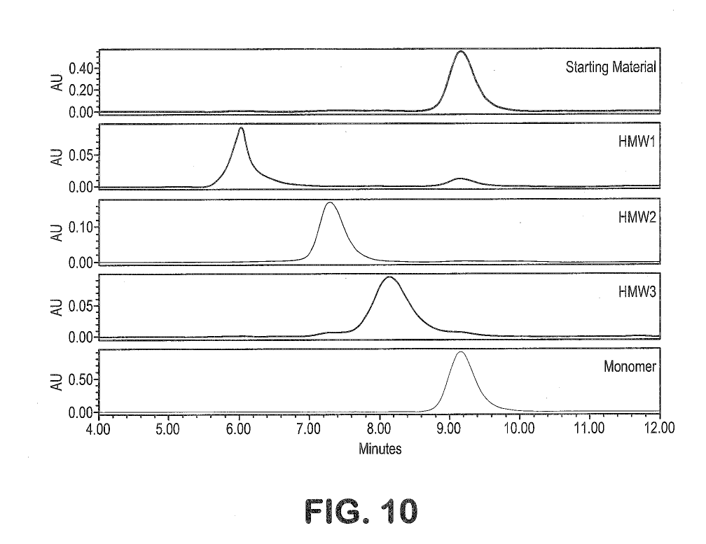

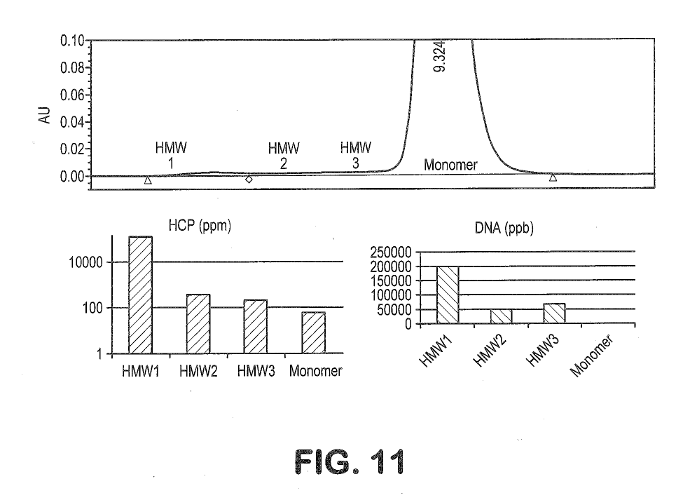

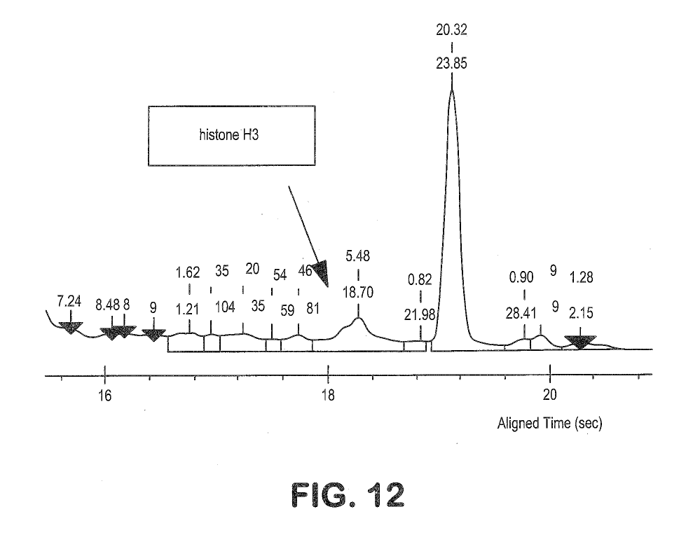

The post protein A elution pools for treated and untreated cell culture showed two distinct size-exclusion chromatographic (SEC) profiles (FIG. 9). The fractionation and characterization of the SEC aggregates from untreated Protein A eluate revealed that the HMW1, with MW of 5000 kDa, contained very high level of HCP and DNA (FIGS. 10 and 11). In addition, the results from chip-based CE-SDS showed a distinct peak with MW of 18 kDa, suggesting the present of histone H3 (FIG. 12). However, the HMW1 was not present in the harvest treated Protein A eluate, suggesting that the chromatin related species have been removed by the harvest treatment.

### Example 9

### Virus Removal

This example demonstrated that the harvest treatment was capable of removing virus from the antibody cell culture by acid precipitation with dextran sulfate.

The study was designed to use PCR to detect endogeneous retrovirus in bioreactor samples, post harvest treatment samples and post depth filtration samples from two pilot plant lots. Consistent LRV (2.24 and 2.39) of endogeneous retrovirus were achieved (Table 4). The major contribution of virus removal should be from harvest treatment instead of depth filter. The potential hypothesis is the charge interaction and co-flocculation between positive-charged virus particles and anionic dextran sulfate at pH 4.8.

TABLE 4
Virus Clearance in harvest treatment

| | | RT-PCR RLP/ml | LRV |
|---|---|---|---|
| Batch A | D14 bioreactor sample | 2.86E+05 | NA |
| | Post-harvest treatment sample | <7.14E+03 | >1.60 |
| | Clarified bulk | 1.64E+03 | 2.24 |
| Batch B | D14 bioreactor sample | 3.00E+05 | NA |
| | Post-harvest treatment sample | <7.14E+04 | >0.62 |
| | Clarified bulk | 1.21E+03 | 2.39 |

### Example 10

### Particular Enzymes Removal

This example demonstrated that the harvest treatment was capable of removing certain enzymes from the antibody cell culture by acid precipitation with dextran sulfate. These enzymes may include certain proteases which may cause protein fragmentation due to proteolysis, or these enzymes such as thioredoxin (Trx) and thioredoxin reductase (TrxR) which may cause low molecular weight formation due to disulfide reduction. The free thiol level, which was an indication of the extent of reducing environment, was also measured.

The study was designed to evaluate the level of enzymes (Trx and TrxR) before and after the harvest treatment, using Cayman's thioredoxin reductase colometric assay kit. It was based on the reduction of DTNB (Ellman's reagent: 5,5′-dithio-bis(2-dinitrobenzoic acid)) with NADPH to 5-thio-2-nitrobenzoic acid (TNB) which produced a yellow product that was measured at 412 nm.

TrxR+DTNB+NADPH+H⁺→2TNB+NADP⁺

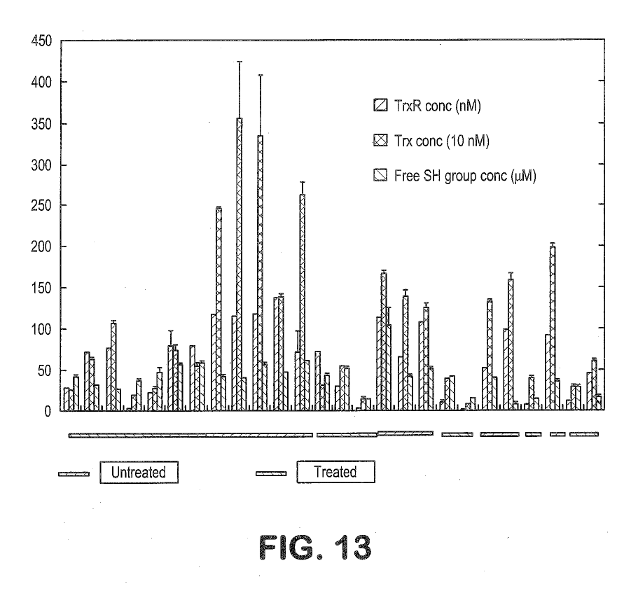

Samples from multiple bioreactors that were used to produce antibody A underwent harvest treatment using low pH with dextran sulfate. The pre and post treatment samples were tested for Trx, TrxR and free —SH using the assay described above. The results were plotted in FIG. 13, which showed that the harvest treatment was capable of removing Trx and TrxR, which could potentially cause disulfide reduction to form low molecular weight species. The relative lower free thiol level in the treated cell cultures was a direct reflection of decrease of reducing power probably due to Trx/TrxR removal.

### EQUIVALENTS

Those skilled in the art will recognize or be able to ascertain using no more than routine experimentation, many equivalents of the specific embodiments of the invention described herein. Such equivalents are intended to be encompassed by the following claims.

### INCORPORATION BY REFERENCE

All patents, pending patent applications, and other publications cited herein are hereby incorporated by reference in their entireties.

1. A method of purifying a protein of interest from a mixture which comprises the protein of interest and one or more contaminants, comprising:
a) lowering the pH of the mixture;
b) adding a dextran polymer to the mixture to form a contaminant precipitate; and
c) separating the contaminant precipitate from the mixture to form a solution, thereby purifying the protein of interest.

2. The method of claim 1, wherein the contaminants are selected from host cell proteins, host cell metabolites, host cell constitutive proteins, nucleic acids, enzymes, endotoxins, viruses, product related contaminants, lipids, media additives and media derivatives, antibody aggregates, chromatin, and cell culture additives.

3. The method of claim 1, wherein said dextran polymer is selected from dextran, dextran sulfate, dextran sulfate sodium salt, and DEAE-dextran hydrochloride.

4. The method of claim 3, wherein the molecular weight of dextran polymer ranges from 8 kDa to 500 kDa.

5. The method of claim 1, wherein the amount of the dextran polymer is between 0.01 to 0.5% by the volume of the harvest.

6. The method of claim 1, wherein the mixture is selected from a cell culture, a harvested cell culture fluid, a cell culture supernatant, a conditioned cell culture supernatant, a cell lysate, and a clarified bulk.

7. The method of claim 6, wherein the cell culture is a mammalian cell culture or a microbial cell culture.

8. The method of claim 6, wherein the cell culture is a Chinese Hamster Ovary (CHO) cell culture.

9. The method of claim 6, wherein the cell culture is in a bioreactor.

10. The method of claim 1, wherein the pH of the mixture is between about 3.0 and about 8.0.

11. The method of claim 1, wherein the contaminant precipitate is separated from the mixture by centrifugation, depth filtration or tangential flow filtration.

12. The method of claim 1, wherein the pH is lowered before, during or after the addition of the dextran polymer.

13. The method of claim 1, wherein the pH is lowered to a pH ranging from about 3.0 to about 6.5.

14. The method of claim 1, further comprising subjecting the solution to a first chromatography.

15. The method of claim 14, wherein the first chromatography is selected from the group consisting of ion exchange, hydrophobic interaction, affinity, mimetic, and mixed mode.

16. The method of claim 1, wherein the protein of interest is an antibody or an Fc fusion protein.

17. The method of claim 16, wherein the antibody is a monoclonal antibody.

18. A method of purifying a protein of interest from a mixture which comprises the antibody and one or more contaminants, comprising:
a) contacting the mixture with a dextran polymer under conditions suitable for the dextran polymer to bind to one or more contaminants, thereby to form a contaminant precipitate;
b) separating the contaminant precipitate from the mixture to form a solution, thereby purifying the protein of interest.

19. The method of claim 18, wherein the conditions comprise lowering the pH of the mixture before, during or after the addition of the dextran polymer.

20. The method of claim 18, wherein the mixture comprises a feedstock.
